(19) United States
(12) Patent Application Publication
(10) Pub. No.: US 2023/0027912 A1
(43) Pub. Date: Jan. 26, 2023

(54) DEEP-LAYER REMINERALIZATION OF HYDROXYLAPATITE

(71) Applicant: DR. KURT WOLFF GMBH & CO. KG, Bielefeld (DE)

(72) Inventors: Joachim ENAX, Bielefeld (DE); Frederic MEYER, Bielefeld (DE); Erik SCHULZE ZUR WIESCHE, Bielefeld (DE)

(73) Assignee: DR. KURT WOLFF GMBH & CO. KG, Bielefeld (DE)

(21) Appl. No.: 17/782,566

(22) PCT Filed: Dec. 4, 2019
(86) PCT No.: PCT/EP2020/084748

§ 371 Date: Jun. 3, 2022

(30) Foreign Application Priority Data
Dec. 5, 2019 (EP) 19213949.1
Mar. 19, 2020 (DE) 10 2020 001 823.8

(51) Int. Cl. A61K 8/24; C01B 25/32; A61Q 11/00

(52) U.S. Cl. CPC A61K 8/24; C01B 25/32; A61Q 11/00; C01P 2004/50

(57) ABSTRACT

The present invention relates to the use of Ca₅(PO₄)₃(OH) (hydroxyapatite; HAP) and a dental care composition comprising Ca₅(PO₄)₃(OH) for the deep-layer remineralization of demineralized teeth, in particular for the deep-layer remineralization of demineralized dental enamel. Ca₅(PO₄)₃(OH) used according to the invention and the dental care composition used according to the invention can be applied in the treatment and/or prevention of various diseases affecting the teeth, in particular caries.

The present invention relates to the use of Ca₅(PO₄)₃(OH) (hydroxyapatite; HAP) and a dental care composition comprising Ca₅(PO₄)₃(OH) for the deep-layer remineralization of demineralized teeth, in particular for the deep-layer rem ineralization of demineralized dental enamel. Ca₅(PO₄)₃(OH) used according to the invention and the dental care composition used according to the invention can be applied in the treatment and/or prevention of various diseases affecting the teeth, in particular caries.

Dental care is becoming more and more important simply because of the increasingly carbohydrate-rich diet worldwide. In addition to aesthetic aspects, special importance is increasingly attached to preventive care, wherein the main focus here is the reduction or even avoidance of plaque, caries, and/or halitosis (bad breath) and healthy gums.

The gums are distinguished, among other things, in that they cervically surround the teeth. The gums enclose the tooth neck, by which the entry point of the tooth into the jawbone of the oral cavity is sealed. The gums are thus used, among other things, to protect and hold the tooth.

The various parts of a natural tooth are the tooth crown, tooth neck, and tooth root, wherein these are constructed from multiple layers. Of these layers, one normally only sees the outer tooth enamel (enamel), which encloses the dentin and further layers. For example, in order to be able to bite or grind food without damaging the teeth, the tooth enamel is very hard. It consists of approximately 97 wt. % of hydroxyapatite, which has the molecular formula Ca₅(PO₄)₃(OH). The dentin is also considered to be a hard tooth substance and also consists of about two-thirds of hydroxyapatite. In addition to hydroxyapatite, however, dentin also contains proteins and water and is therefore not as hard as tooth enamel.

Dental diseases such as caries can originate from the formation of bacterial microfilms and/or bacterial inflammation. Although it is actually often avoidable by preventive care, caries remains one of the most common chronic diseases in children, for example in the USA, and there is a great need for products for healing teeth affected by caries, especially among poorer children worldwide.

In addition to caries, there are other dental diseases such as tooth erosion, bruxism, and amelogenis imperfecta. In addition, molar incisor hypomineralization (MIH), for example, is widespread and a growing problem worldwide. According to the Fifth German Oral Health Study (DMSV), 28.7% of 12-year-olds in the country are affected and have at least one tooth affected by molar incisive hypomineralization (MIH), while in the comparable group of people, 18.7% have caries lesions.

It is documented that the saliva, due to its oversaturation of Ca²⁺ and PO₄³⁻ ions in a bioavailable form and its enrichment in various proteins important for maintaining the integrity of hard tissues, has a caries-protective effect. In particular, oversaturation of the saliva with Ca²⁺ and PO₄³⁻ ions at physiological pH ensures that these bioavailable ions can diffuse into tooth lesions having insufficient mineralization to induce remineralization there. However, the caries-protective and remineralizing effect brought about by the saliva is not only slow, but also apparently insufficient to protect people from caries and/or to remineralize existing tooth lesions without the addition of remineralization-enhancing additives.

The use of fluoride-containing dental care preparations appears to be suitable both to prevent the spread of caries and to remineralize initial tooth lesions. Various fluoride compounds such as sodium fluoride, tin fluoride, amine fluorides, and monofluorophosphates are suitable as fluoride sources. However, there seem to be limits to the use of fluoride alone in preventing and/or treating caries and rem ineralization. It is reported that there is still a risk of caries formation in people of all ages despite the administration of fluoride, and that in addition to the provision of fluoride, bioavailable Ca²⁺ and PO₄³⁻ ions from the saliva are necessary to achieve the above-mentioned effects. Furthermore, using the commercially available dental care products containing fluoride, remineralization in the outer region of the lesion (up to 30 μm) is achieved. In other words, fluoride-containing dental care products are essentially most effective at rem ineralization of a lesion of up to 30 μm. Such rem ineralization can be considered to be surface remineralization, which can be at the expense of and even make it difficult for complete remineralization of a lesion.

It is known that the effectiveness of fluorides, for example in remineralization, increases with their dosage. However, the concentration of fluorides in dental care products is limited, since the risk of undesirable side effects increases if the dosage is excessively high. One of these is so-called fluorosis, which is caused by excessive fluoride intake and has symptoms such as nausea, vomiting, and diarrhea. Other examples are bone fluorosis, which manifests itself through thickening of the outer bone layer and the associated loss of elasticity and resilience of the bones, and enamel fluorosis, which can be recognized by the appearance of whitish enamel spots on the tooth surface. In addition, it has been reported that ingestion of dental care products with concentrations of fluoride below the regulatory concentration of 1000 to 1500 ppm for non-prescription dental care products can cause acute fluoride poisoning in children under 6 years of age, which can sometimes even be fatal.

For these reasons, the concentration of fluorides actually required for remineralization often has to be avoided and a lower, suboptimal concentration has to be used for remineralization.

Due to the above limitations in both the homeostatic mechanism due to the saliva and also in the approaches based on fluoride supply to caries prevention and remineralization, there is a need for alternative strategies that are at least equivalent to the efficiency of fluoride supply in remineralization, but without displaying the corresponding undesired side effects.

Biomimetically acting tooth and mouthwash solutions having artificial tooth enamel can contain, for example, zinc carbonate hydroxyapatite. This zinc carbonate hydroxyapatite is also known commercially as Microrepair. There are also biometric dental care products based on the use of hydroxyapatite. For example, DE 10 2016 114 189 and DE 10 2018 102 365 describe oral care compositions containing synthetic hydroxyapatite, wherein hydroxyapatite, as indicated above, is a bioactive and biocompatible material having a similar chemical composition to the apatite of human tooth enamel. However, it is unanimously disclosed in the prior art (see also WO 2015/074240 A1) that hydroxyapatite only accumulates superficially on the tooth enamel surface. This has been clearly confirmed by various studies (ex-in vivo, in situ, in vitro) by diverse research groups in different study designs (Lelli, M. et al. Remineralization and repair of enamel surface by biomimetic Zn-carbonate hydroxyapatite containing toothpaste: a comparative in vivo study. Front. Physiol. 5, 333 (2014) and Kensche, A. et al. Efficacy of a mouthrinse based on hydroxyapatite to reduce initial bacterial colonisation in situ. Arch. Oral Biol. 80, 18-26 (2017)).

Thus, there is still a need for the use of a product to treat or prevent diseases affecting the teeth, wherein this product can also be used for remineralization of lesions lying under the surface region. In other words, the use of the product is not only to provide an opportunity for the prevention or treatment of numerous dental diseases, in particular caries, but is also to provide a remineralization in deeper layers or cavities of the tooth/dental enamel (deep-layer remineralization), which cannot be ensured using conventional products, in particular those containing fluoride.

Furthermore, when using the product, the ecological equilibrium in the oral region is not to be significantly disturbed and/or tooth discoloration or taste disturbance is not to be risked.

These objects are achieved by the present application according to the claims.

In particular, it was unexpectedly possible to establish that the use according to the invention of Ca₅(PO₄)₃(OH) and/or a dental care composition comprising Ca₅(PO₄)₃(OH) can prevent caries and also lesions down to the deeper layers of the tooth, in particular the tooth enamel, can be remineralized. Furthermore, by way of the use according to the invention of Ca₅(PO₄)₃(OH) or a dental care composition comprising Ca₅(PO₄)₃(OH), a protective layer can be applied to the tooth and over exposed dentin and, in particular, open dentin tubules can be closed. Furthermore, it was found that the tooth enamel after the application has a significantly reduced or no longer detectable structural damage. In addition, the application of fluoride can be dispensed with in the present use. In this way, the above positive aspects can be guaranteed without, for example, disturbing/destroying the bacterial equilibrium in the oral cavity and without risking undesirable side effects, which can occur, for example, when using fluoride-containing oral care products.

The subject matter of the present invention is the use of Ca₅(PO₄)₃OH for the remineralization of demineralized teeth, in particular demineralized dental/tooth enamel, not only superficially but to a depth of 200 μm, preferably to 100 μm (deep mineralization).

Ca₅(PO₄)₃(OH) is also known as hydroxyapatite and more rarely hydroxylapatite. It is a mineral from the mineral class of phosphates, which crystallizes in a hexagonal crystal system. In addition, hydroxyapatite is a member of the apatite group and, with chloroapatite and fluoroapatite, forms a continuous mixed series.

The Ca₅(PO₄)₃(OH) used according to the invention is preferably produced synthetically. This means that the Ca₅(PO₄)₃(OH) used according to the invention is preferably not obtained by burning out the organic components from animal material, for example bones.

In a preferred embodiment, the Ca₅(PO₄)₃(OH) used according to the invention is provided in pure form. According to the invention, a pure form is present when the ions (Ca²⁺, PO₄³⁻ and OH⁻) contained in Ca₅(PO₄)₃(OH) are each substituted less than 1%, preferably less than 0.5%, still more preferably less than 0.1% by one or more other ions. For example, in pure hydroxyapatite, the Ca²⁺ ions are substituted by, for example, Mg²⁺ or Zn²⁺ less than 1%, preferably less than 0.5%, still more preferably less than 0.1%. Furthermore, the hydroxylapatite used according to the invention preferably contains no doping, for example, a zinc carbonate doping.

In a preferred embodiment of the invention, the Ca₅(PO₄)₃(OH) used according to the invention is provided in crystalline form. Thus, the Ca₅(PO₄)₃(OH) preferably has a hexagonal crystal lattice in which the length of the a axis is 0.930 to 0.950 nm, preferably 0.933 to 0.948 nm, particularly preferably 0.936 to 0.945 nm and the length of the c axis is 0.680 to 0.700 nm, preferably 0.682 to 0.696 nm, particularly preferably 0.685 to 0.692 nm. The lengths of the a axis and the c axis are determined by a Rietveld evaluation of the corresponding X-ray powder diffractograms. The X-ray powder diffractograms themselves are obtained by means of a measurement using a conventional powder diffractometer at the routine settings.

It is furthermore preferred that the Ca₅(PO₄)₃(OH) used according to the invention has a largely spherical crystal morphology.

In a preferred embodiment of the invention, the Ca₅(PO₄)₃(OH) used according to the invention is provided in aggregated form. In this case, aggregation is understood to mean an agglomeration of molecules or particles to form a larger group, the aggregate. This aggregation or aggregate is caused and held together by various forces and/or types of bonds such as ionic bonds, van der Waals forces, intermolecular forces, or other types of chemical bonds. The degree of aggregation and also the size of the aggregate can be determined with the aid of scanning electron microscopy. It is preferred that in the case of Ca₅(PO₄)₃(OH) in aggregated form, no nanoparticles can be detected even after high energy input. Particles having a size of less than 100 nm are referred to as nanoparticles. In a particularly preferred embodiment, the Ca₅(PO₄)₃(OH) used according to the invention is provided in microaggregated form. Microaggregated form is understood to mean microclusters of Ca₅(PO₄)₃(OH) crystals, wherein these microclusters have a preferably relatively uniform form having a length of preferably 60 to 100 nm, in particular approximately 80 nm, and a width of preferably 20 to 40 nm, in particular approximately 30 nm.

A Ca₅(PO₄)₃(OH) suitable according to the invention is described, for example, in DE 10 2016 114 189.5.

The Ca₅(PO₄)₃(OH) used according to the invention is used for the remineralization of teeth to a depth of 200 μm, preferably to 150 μm, in particular to 100 μm. Such a remineralization of teeth down to these depths is referred to as deep mineralization, since in this case not only the tooth surface regions are remineralized down to a depth of approx. 30 μm, as described in the prior art, but also deeper regions of the tooth. In contrast to the remineralization of tooth surface regions, as described, for example, in WO 2015/074240 Al (see FIG. 1(c) therein), which discloses remineralization/adhesion in the nanometer range and in particular not in the micrometer range), according to the invention and surprisingly, there is not in any way an accumulation on the tooth enamel surface by adhesion. Rather, the hydroxyapatite used according to the invention itself remineralizes deeper layers of the demineralized tooth enamel. The remineralization therefore preferably does not take place superficially, but exclusively in a depth range of preferably 200 μm to 50 μm, more preferably from 200 μm to 60 μm or 150 μm to 60 μm, and particularly preferably from 200 μm to 75 μm or 150 μm to 75 μm or 100 μm to 75 μm.

It was unexpectedly found that the Ca₅(PO₄)₃(OH) used according to the invention can be used in the treatment and/or prevention of numerous dental diseases.

In a preferred embodiment of the invention, the Ca₅(PO₄)₃(OH) used according to the invention can be used for the treatment of (dental) diseases/conditions selected from caries, tooth erosion, tooth abrasion, attrition, bruxism, molar incisor hypomineralization (MIH), amelogenesis imperfecta, dentinogenesis imperfecta, and fluorosis.

The term caries is familiar to those skilled in the art. Thus, caries is generally understood to be a destructive disease of dental hard tissue, tooth enamel, and dentin.

Tooth erosion is understood to mean damage to the hard tooth substance by acids, in other words defects in the tooth enamel and/or dentin due to dental erosions which, if treated too late, can result in irreversible damage.

(Tooth) abrasion is understood to mean the loss of hard tooth substance by friction. Attrition is a sub-form of abrasion, namely the loss of hard tooth substance by reflectory touching of the teeth.

Bruxism is the unconscious grinding or clenching of teeth, mostly at night, but also during the day, with the result that not only the teeth but also the periodontium and chewing muscles can be worn out.

Molar incisive hypomineralization (MIH), also known under the name “chalk teeth”, is an enamel formation disorder, i.e., structural damage to the tooth enamel.

Amelogenesis imperfecta is considered to be a genetic disease in which tooth enamel formation is disrupted. As a result, the teeth have an increased risk of caries formation and are particularly sensitive to temperature.

Dentinogenesis imperfecta is a malformation/structural disturbance, inherited in an autosomal dominant manner, of the tooth dentition, which affects approximately 1 in 8000 people and causes severe abrasion of the teeth.

Tooth (fluorosis) (also: dental fluorosis) is understood to be a non-inflammatory disease (“mottled teeth”), which is caused by excessive fluoride intake, particularly during the ontogenetic development of the teeth.

It has been found by way of the use according to the invention, the above dental (diseases) can be avoided and/or their course can at least be significantly slowed and/or complete restoration of the tooth substance, in particular the hard tooth substance, can be achieved. In particular, it has been found that by way of the use according to the invention or after its application, the tooth enamel has significantly reduced or no longer detectable structural damage.

In a preferred embodiment of the invention, the caries is a code 3 or code 4 caries, preferably a code 3 caries; defined according to the International Caries Detection and Assessment System (ICDAS).

According to the International Caries Detection and Assessment System (ICDAS), caries is divided into different codes (levels), wherein the higher the code, the greater the caries infestation on the tooth and consequently its effects on this tooth.

In the case of code 0 caries, no signs of caries are visible after drying in the air stream for approximately 5 seconds.

In the case of code 1 caries, the first visual changes in the enamel surface are visible after the tooth has dried. The changes can be opacity or whitish or brownish discoloration.

In the case of code 2 caries, clear visual changes in the enamel surface are already present on the moist tooth. These changes can cause opacities in the sense of a white spot lesion and/or brownish carious discoloration in the fissures/trenches and still have to be visible on the dried tooth.

In the case of code 3 caries, there is demineralization or loss of the enamel structure without visible changes in the dentin. The opacities and/or brownish or black carious changes extend beyond the border of fissures/trenches and are visible even after the tooth has dried. If necessary, a WHO probe can be carefully passed over the enamel defect to palpate the discontinuity of the enamel surface.

In the case of code 4 caries, there is shadowing in the dentin, with or without enamel collapse. Shadowing can be greyish, bluish, or brownish.

In the case of code 5 caries, clear cavity formation with visible dentin is recognizable. The loss of enamel is clearly visible on the dried tooth. If necessary, the WHO probe can be used to palpate the exposed dentin.

In the case of code 6 caries, there is extensive cavitation, wherein the dentin is clearly visible in the width and depth of the tooth. At least half of the enamel surface has been destroyed by caries. The pulp can be affected.

In a preferred embodiment, the use according to the invention of Ca₅(PO₄)₃OH can be a cosmetic and/or medical use. This means that it can be used not only for the treatment of the (dental) diseases mentioned above, but also for cosmetic purposes such as improving the appearance of the teeth.

In a preferred embodiment, the Ca₅(PO₄)₃OH used according to the invention is used in children, preferably in children aged 6 months to 14 years, in particular in children aged 10 months to 12 years.

A further object of the present invention is the use of a dental care composition comprising Ca₅(PO₄)₃OH for the remineralization of teeth, in particular tooth enamel, to a depth of 200 μm, preferably to 100 μm.

Just like the Ca₅(PO₄)₃(OH) used according to the invention, the dental care composition used according to the invention can be used in the treatment and/or prevention of numerous dental diseases.

In a preferred embodiment, the dental care composition used according to the invention can be used for the treatment of (dental) diseases selected from caries, tooth erosion, tooth abrasion, attrition, bruxism, molar incisor hypomineralization (MIH), amelogenesis imperfecta, dentinogenesis imperfecta, and fluorosis.

With regard to (dental) diseases, the same applies as described above.

In a preferred embodiment, the dental care composition used according to the invention can be used to treat code 3 or code 4 caries, preferably code 3 caries; defined according to the International Caries Detection and Assessment System (ICDAS).

In a preferred embodiment, the Ca₅(PO₄)₃OH used according to the invention is used in children, preferably in children aged 6 months to 14 years, in particular in children aged 10 months to 12 years.

The same as described above applies to the Ca₅(PO₄)₃OH contained in the dental care composition used according to the invention and to the remineralization. For example, the hydroxylapatite comprised by the dental care composition used according to the invention is preferably a synthetically produced Ca₅(PO₄)₃OH and/or is preferably provided in aggregated, more preferably in microaggregated form.

In a preferred embodiment, the Ca₅(PO₄)₃OH comprised by the dental care composition used according to the invention is the sole apatite component of the dental care composition.

In a preferred embodiment, the dental care composition used according to the invention comprises 0.1 to 50 wt. %, preferably 0.2 to 40 wt. %, more preferably 0.5 to 30 wt. %, in particular 1.0 to 20 wt. % Ca₅(PO₄)₃(OH). In a preferred embodiment of the invention, the dental care composition used according to the invention can comprise 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 20 wt %, or 25 wt % Ca₅(PO₄)₃(OH).

In a preferred embodiment, the dental care composition used according to the invention comprises one or more calcium salts which have a solubility of at least 10 mg/L H₂O at 20° C. The solubility is determined according to methods known to those skilled in the art or can be found in the relevant specialist literature.

In a preferred embodiment, the composition used according to the invention comprises 0.0001 to 40 wt. %, preferably 0.001 to 30 wt. %, more preferably 0.0025 to 20 wt. %, more preferably 0.005 to 10 wt. %, in particular 0.01 to 7 wt. % of one or more calcium salts having a solubility of at least 10 mg/L H₂O at 20° C. In a preferred embodiment of the invention, the composition can comprise 0.005 wt. %, 0.01 wt. %, 0.025 wt. %, 0.05 wt. %, 0.1 wt. %, 0.25 wt. %, 0.5 wt. %, 1 wt. %, 2 wt. %, 2.5 wt. %, 3 wt. %, 4 wt. %, 5 wt. %, 7 wt. %, 10 wt. %, or 25 wt. % of one or more calcium salts having a solubility of at least 10 mg/L H₂O at 20° C.

Examples of calcium salts having a solubility of at least 10 mg/L H₂O at 20° C. are calcium carbonate, calcium chloride, calcium bromide, calcium sulfate, calcium phosphate, calcium nitrate, calcium acetate, calcium gluconate, calcium lactate, calcium tartrate, and their hydrates and mixtures thereof.

In a preferred embodiment of the invention, the one or more calcium salts having a solubility of at least 10 mg/L H₂O at 20° C. are selected from calcium carbonate, calcium chloride, calcium bromide, calcium nitrate, calcium acetate, calcium gluconate, calcium lactate, calcium tartrate, and their hydrates and mixtures thereof.

In a preferred embodiment of the invention, the one or more calcium salts b) having a solubility of at least 10 mg/L H₂O at 20° C. are selected from calcium acetate, calcium gluconate, calcium lactate, and their hydrates and mixtures thereof.

In an alternative preferred embodiment, the one or more calcium salts having a solubility of at least 10 mg/L H₂O at 20° C. are selected from calcium carbonate, calcium chloride, calcium bromide, their hydrates and mixtures thereof. The calcium salt b) is particularly preferably calcium carbonate or a hydrate thereof.

In a preferred embodiment, in the dental care composition used according to the invention, the weight ratio of component (a), Ca₅(PO₄)₃(OH), to component (b), one or more calcium salts having a solubility of at least 10mg/L H₂O at 20° C., is 1:50 to 100:1, preferably 1:10 to 50:1, more preferably 1:5 to 25:1, in particular 1:1 to 20:1, especially 4:1 to 10:1.

In the dental care composition used according to the invention, the weight ratio of component (a), Ca₅(PO₄)₃(OH), to component (b), one or more calcium salts having a solubility of at least 10mg/L H₂O at 20° C. is preferably 1:50, 1:25, 1:10, 1:5; 1:3, 1:1; 2:1, 3:1, 4:1, 5:1, 6:1, 7:1, 8:1, 10:1, 12:1, 15:1, 20:1, 25:1, 30:1, 40:1, 50:1, 60:1, 75:1, or 1000:1.

In a preferred embodiment, the dental care composition used according to the invention can contain one or more pharmaceutical or cosmetic ingredients. These pharmaceutical or cosmetic ingredients are described, for example, in Toothpastes, Monographs in Oral Science, Vol. 23, 1^{st} edition, 2013.

Preferably, the one or more pharmaceutical or cosmetic ingredients include xylitol, antimicrobials, pH regulators, abrasives, flavorings, and humectants, in particular xylitol, pH regulators, abrasives, and flavorings.

Xylitol can minimize the number of caries bacteria and inhibit their growth. Xylitol can also stimulate salivation. The increased amount of saliva produces an increased amount of phosphate. This phosphate can react with the calcium (ions) from the dental care composition used according to the invention to form hydroxyapatite. The dental care composition used according to the invention can contain xylitol in an amount of 0.5 to 15 wt. %, preferably 1 to 10 wt. %, in particular approximately 7.0 wt. %, in relation to the total weight of the dental care composition used according to the invention. In addition to xylitol, the dental care composition used according to the invention can contain further sugar alcohols such as sorbitol.

Antimicrobial substances are substances which can kill microorganisms, such as bacteria, or greatly reduce their reproduction. In addition to antimicrobial substances having a non-specific defense against bacteria and fungi, there are also those that only work against targeted bacteria, for example. The use of antimicrobial substances can also combat bad breath, for example. Preferably, antimicrobial substances can be contained in an amount of 0.01 to 2.0 wt. %, preferably 0.05 to 1.0 wt. % in the dental care composition used according to the invention. Examples of the antimicrobial substances used in oral care are zinc compounds such as zinc chloride and zinc citrate, as well as cetylpyridinium chloride, essential oils, and surfactants.

In a particularly preferred embodiment, the dental care composition used according to the invention does not contain chlorhexidine.

In a particularly preferred embodiment, the dental care composition used according to the invention does not contain triclosane.

In an alternative preferred embodiment, the dental care composition used according to the invention does not contain fluoride and is therefore fluoride-free.

In an alternative preferred embodiment, the dental care composition used according to the invention is free of fluoride and/or free of chlorhexidine and/or free of triclosane.

pH regulators are substances that can set a specific pH range, preferably a range from pH 6.5 to 7.5. This is because if the composition were too acidic, there would be a risk of demineralization of the hard tooth substance (erosion). Examples of pH regulators are sodium hydroxide (NaOH) or phosphoric acid (H₃PO₄), which can be used in accordance with the desired pH value. Sodium hydroxide can be added to raise too low a pH value, while phosphoric acid can be added if the pH value is too high. In relation to the total weight of the composition used according to the invention, pH regulators can be contained up to 5 wt. %.

Abrasives, also known as cleaning or grinding substances, remove plaque and harmful bacteria from the tooth surface during the tooth cleaning process, usually together with the toothbrush, and can also ensure whitening. Abrasives can preferably be contained in the dental care composition used according to the invention in an amount of up to 15 wt. %, preferably 0.1 to 12 wt. %, in relation to the total weight of the composition used according to the invention. Examples of abrasives are whitewash, marble powder, and/or silicate compounds such as silica.

In a preferred embodiment, the dental care composition used according to the invention contains one or more flavorings which can impart the desired taste to the composition used according to the invention. These one or more flavorings can be natural, nature-identical, synthetic flavorings, and/or mixtures thereof Examples of flavorings are limonene, geraniol, citronellol, and eugenol. In addition, flavorings can stimulate salivation, wherein the moisture in the saliva can have a positive effect on the remineralization of the tooth. An example of a salivary flavorant is pellitorin, in particular trans-pellitorin.

Flavorings can preferably be contained in the dental care composition used according to the invention in an amount of up to 0 to 5 wt. %, preferably 0.1 to 3 wt. %, in relation to the total weight of the composition used according to the invention.

Humectants are additives that prevent the composition used according to the invention from drying out by binding water added during production (i.e., preventing evaporation) or by attracting atmospheric moistureduring storage.

Examples of humectants are glycerin, propane-1,2-diol, hexane-1,2-diol, egg yolk, aloe vera gel, honey, molasses, in particular glycerin and hexane-1,2-diol.

Humectants can preferably be contained in the dental care composition used according to the invention in an amount of up to 0 to 25 wt. %, preferably 0.1 to 20 wt. %, in relation to the total weight of the composition used according to the invention.

In a preferred embodiment, the use of the dental care composition according to the invention can be a cosmetic and/or medical use. In other words, the use of the dental care composition according to the invention can be comprise the use in the cosmetic and/or medical field. The cosmetics and/or dental care compositions used in the use according to the invention can be provided in any form known to those skilled in the art, preferably in the form of a toothpaste, oral gel, mouthwash, lozenge, chewing gum, foam, or spray, more preferably in the form of a toothpaste.

Toothpaste, also known as tooth paste, can be used to mechanically clean teeth and is a soft or semi-solid composition for oral application, in particular on the teeth and/or gums.

In a preferred embodiment, the dental care composition used according to the invention is provided as a toothpaste and contains
0.1 to 50 wt. %, preferably 0.2 to 40 wt. %, more preferably 0.5 to 30 wt. %, in particular 1.01 to 20 wt. % Caₛ(PO₄)₃(OH)
0.5 to 15 wt. %, preferably 1 to 10 wt. %, in particular approximately 7.0 wt. % xylitol
0 to 15 wt. %, preferably 0.1 to 12 wt. % abrasive, in particular silica and/or hydrated silica (hydrated silicic acid)
0 to 2.0 wt. %, preferably 0.01 to 2.0 wt. %, more preferably 0.05 to 1.0 wt. % antimicrobial substance, in particular cetylpyridinium chloride and/or zinc chloride,
0 to 5 wt. %, preferably 0.3 to 2.0 wt. % pH regulator, in particular phosphoric acid,
0 to 5 wt. %, preferably 0.1 to 3 wt. % flavoring
from 0 to 25 wt. %, preferably 0.1 to 20 wt. % humectant, in particular glycerin and/or hexane-1,2-diol

wherein the specifications in wt. % relate to the total weight of the dental care composition. The remainder is distilled water, if necessary. The pH is in a neutral range from pH 6.5 to 7.5.

The subject matter of the present invention comprises the following embodiments:

1) Ca₅(PO₄)₃(OH) (hydroxyapatite; HAP) for use in the treatment of at least one disease selected from caries, tooth erosion, tooth wear, tooth abrasion, bruxism, molar incisor hypomineralization (MIH), amelogenesis imperfecta, dentinogenesis imperfecta, and fluorosis.

2) Ca₅(PO₄)₃(OH) for use according to item 1), wherein the caries is a code 3 or code 4 caries according to the International Caries Detection and Assessment System (ICDAS).

3) Ca₅(PO₄)₃(OH) for use according to item 1) or 2), wherein the caries is a code 3 caries according to the International Caries Detection and Assessment System (ICDAS).

4) Ca₅(PO₄)₃(OH) for use according to any one of preceding items 1) to 3) above in children aged between 6 months and 14 years.

5) Ca₅(PO₄)₃(OH) for use according to any one of preceding items 1) to 4), wherein Ca₅(PO₄)₃(OH) is provided in aggregated, preferably in microaggregated, form.

6) (Cosmetic/non-therapeutic) use of Ca₅(PO₄)₃(OH) for remineralization of teeth to a depth of 200 μm, preferably to 100 μm.

7) Use according to point 6), wherein Ca₅(PO₄)₃(OH) is provided in aggregated, preferably in microaggregated, form.

8) Dental care composition comprising Ca₅(PO₄)₃(OH) (h_{y}droxyapatite; HAP) for use in the treatment of at least one disease selected from caries, tooth erosion, tooth wear, tooth abrasion, bruxism, molar incisor hypomineralization (MIN), amelogenesis imperfecta, dentinogenesis imperfecta, and fluorosis.

9) Dental care composition comprising Caₛ(PO₄)₃(OH) for use according to item 8), wherein the caries is a code 3 or code 4 caries according to the International Caries Detection and Assessment System (ICDAS).

10) Dental care composition Ca₅(PO₄)₃(OH) for use according to item 8) or 9), wherein the caries is a code 3 caries according to the International Caries Detection and Assessment System (ICDAS).

11) Dental care composition comprising Ca₅(PO₄)₃(OH) for use according to any one of preceding items 8) to 10) in children aged between 6 months and 14 years.

12) Dental care composition comprising Ca₅(PO₄)₃(OH) for use according to any one of preceding items 8) to 11), wherein Ca₅(PO₄)₃(OH) is provided in aggregated, preferably in microaggregated, form.

13) Dental care composition comprising Ca₅(PO₄)₃(OH) for use according to any one of preceding items 8) to 12), wherein the dental care composition is a toothpaste, oral gel, mouthwash, lozenge, chewing gum, foam, or spray.

14) Dental care composition comprising Ca₅(PO₄)₃(OH) for use according to any one of the preceding items 8) to 13), wherein the dental care composition comprises Ca₅(PO₄)₃(OH) in an amount of 0.1 to 50.0 wt. %, preferably 0.1 to 20.0 wt. %, in relation to the total weight of the dental care composition.

15) Dental care composition comprising Ca₅(PO₄)₃(OH) for use according to any one of preceding items 8) to 14), wherein the dental care composition comprises one or more pharmaceutical or cosmetic ingredients.

16) Dental care composition comprising Ca₅(PO₄)₃(OH) for use according to item 15), wherein the one or more pharmaceutical or cosmetic ingredients comprise pH regulators, xylitol, abrasives, and flavorings.

17) Dental care composition comprising Ca₅(PO₄)₃(OH) for use according to any one of preceding items 8) to 16, wherein the dental care composition is free of fluoride.

18) (Cosmetic/non-therapeutic) use of a dental care composition comprising Ca₅(PO₄)₃(OH) for the remineralization of teeth to a depth of 200 μm, preferably to 100 μm.

19) Use according to item 18), wherein Ca₅(PO₄)₃(OH) is provided in aggregated, preferably in microaggregated, form.

20) Use according to item 18) or 19), wherein the dental care composition is a toothpaste, oral gel, mouthwash, lozenge, chewing gum, foam, or spray.

21) Dental care composition comprising for use according to any one of the preceding items 18) to 20), wherein the dental care composition comprises Ca₅(PO₄)₃(OH) in an amount of 0.1 to 50.0 wt. %, preferably 0.1 to 20.0 wt. %, in relation to the total weight of the dental care composition.

22) Use according to any one of preceding items 18) to 21), wherein the dental care composition comprises one or more pharmaceutical or cosmetic ingredients.

23) Use according to item 22), wherein the one or more pharmaceutical or cosmetic ingredients comprise pH regulators, xylitol, abrasives, and flavorings.

24) Use according to any one of the preceding items 18) to 23, wherein the dental care composition is free of fluoride.

The invention is explained below using examples.

### EXAMPLE 1

A toothpaste was produced which contains the following ingredients:

| | |
|---|---|
| Demineralized water | 44.30 wt. % |
| Hydrogenated starch hydrolyzate | 21.60 wt. % |
| Silica hydrated (hydrated silicic acid) | 13.00 wt. % |
| Hydroxyapatite | 10.00 wt. % |
| Xylitol | 7.00 wt. % |
| Silica (highly dispersed) | 2.50 wt. % |
| Sodium carboxymethyl cellulose | 0.80 wt. % |
| Hexane-1,2-diol | 0.40 wt. % |
| Flavor | 0.40 wt. % |

The toothpaste is a light beige, homogeneous, creamy paste having an average pH of 7.2.

Study

A study was made on properties of the dental care composition used according to the invention.

### FIGURES

FIG. 1 shows a flow chart having the step-by-step methodology. This is a crossover study, so the 30 test subjects who completed the study received the two procedures in a crossover design as phase I & II.

FIG. 2 shows representative microradiographic images of healthy dental tissue before (A) and after (B) inter-oral exposure to demineralization, while the test subject uses a children's toothpaste (Karex™) containing 10% hydroxyapatite in the form of microclusters.

FIG. 3 shows representative microradiographic images of healthy tooth tissue before (A) and after (B) inter-oral exposure to demineralization while the test subject uses a children's toothpaste (Elmex) containing 500 ppm fluoride as amine fluoride.

FIG. 4 shows representative microradiographic images of lesions lying below the surface on the tooth enamel (initial carious lesions) before (A) and after (B) in situ remineralization by treatment of a children's toothpaste (Karex™) containing 10% hydroxyapatite in the form of microclusters.

FIG. 5 shows representative microradiographic images of lesions lying below the surface on the tooth enamel (initial carious lesions) before (A) and after (B) in situ remineralization by treatment of a children's toothpaste (Elmex) containing 500 ppm fluoride as amine fluoride.

### NATURE OF THE STUDY

In a randomized, monocentric, in situ controlled, crossover double-blind study, two children's toothpaste compositions containing either 10% hydroxyapatite in the form of microclusters or 500 ppm fluoride provided as amine fluoride (AMF) were compared to one another with respect to their ability to induce rem ineralization and prevent the development of initial carious lesions.

Test Subject Selection

The study included 32 people of various ethnic origins, ages 18 to 60, who were not taking antibiotics or medications that negatively affect salivation, had at least 20 natural, uncapped teeth, and who had a history of caries but who had no clinically active caries at the beginning of the study. In addition, the persons had no dental and/or gum disease, were not pregnant or breastfeeding, and did not smoke tobacco products.

Creation of Artificial Initial Caries and Production of the In Situ Device

32 freshly extracted human deciduous teeth without caries, cracks, or enamel defects were selected and cleaned. Four tooth blocks were produced from the buccal and lingual surfaces of each of the selected teeth, wherein each of the blocks was approximately 2 mm in length, 2 mm in width, and 1.5 mm in depth. Two blocks were retained as healthy blocks for the assessment of the demineralization prevention, while artificial initial caries was induced in the two blocks designated for the assessment of the remineralization assessment. All sides of each block were coated in two coats of acid-resistant nail polish except for the buccal and lingual surfaces, respectively, on which an initial caries lesion (demineralization) was created by subjecting the exposed surface to an acidified gel system (0.1 M lactic acid, 0.1 M sodium hydroxide, 60% w/v hydroxyethyl cellulose, pH 4.5) for seven days. The nail polish was then carefully removed using acetone. A tooth section approximately 150 μm thick was cut from each tooth block to measure the baseline mineral loss (Δz₁) and lesion depth (LD₁) of each induced initial carious lesion and to select the lesions suitable for remineralization evaluation. The sections were prepared for transverse microradiography as follows. Both sides of the sections were polished using a lapping film in an MultiPrep™ precision polishing machine from Allied High Tech to create plane-parallel surfaces and reduce the thickness of the sections to 100 μm. Sections were then microradiographed on a Type 1A high-resolution glass X-ray plate from Microchrome Technology, CA, USA using a Philips X-ray generator having the suitable settings. The plates were exposed to radiation for 10 minutes at an anode voltage of 20 kV and a tube current of 10 mA and then further processed, wherein the further processing consisted of a five-minute development in a Kodak HR developer, a fifteen-minute fix using a fixing agent (Kodak Rapid-fixer), and a thirty-minute wash. After drying, the microradiographs were examined under an optical microscope (Leica DMR) connected to a PC via a camera (Sony; model XC-75 CE CCTV). Using software for image analysis (TMR2006 version 3.0.0.11; Inspector Research Systems, Amsterdam), the magnified image of the microradiographs was analyzed under standard conditions with respect to light intensity and magnification together with the image of a step wedge described in the literature. Then the images were only used to select the lesions suitable for the comparison experiment. Only the samples that had caries-like surface lesions that displayed a reasonably uniform width along their length were selected for the remineralization process. Their blocks were used for the device of the in situ application.

As mentioned above, the four blocks from each tooth were distributed as follows: two blocks having lesions for the assessment of the remineralization and two blocks for the assessment of the demineralization inhibition. These blocks were used to fabricate the in situ device as follows. Each block was covered with a polyester gas (Bard Peripheral Vascular, Inc. Tempe, Ariz., USA) and mounted in a fitted orthodontic fixture. The device consists of an orthodontic molar pad with retaining mesh lining (American Orthodontics Corp., Sheboygan, USA) having a ring of 0.7 mm orthodontic wire bent so that the ring snugly encloses each test block. Each device was sterilized using gamma radiation prior to delivery to the test subject.

Carrying Out the Study

The study was conducted in two different treatment phases, in which the test subjects were subjected to one of the following two treatments in a randomized, crossover comparison: (A) Toothpaste containing 10% hydroxyapatite in the form of microclusters (Kinder Karex™, Dr. Kurt Wolff GmbH & Co. KG, Bielefeld, Germany and (B) toothpaste containing 500 ppm fluoride provided as amine fluoride (AMF) (Elmex children's toothpaste, GABA GmbH, Hamburg, Germany). A one-week rinsing phase was followed by a four-week treatment phase, which consisted of two-week phases during which each test subject applied his/her assigned treatment under the following conditions: the first two-week phase for the test subjects wearing the healthy enamel block in situdevice and the two-week phase for the subjects wearing the in situdevice having enamel block with lesions.

For the one-week rinsing phase, the test subjects who met the inclusion criteria were given a specially made rinsing toothpaste that contained neither hydroxyapatite nor fluoride for two minutes of use twice a day (morning and evening).

After the rinsing phase, the test subjects were assigned to either the group using hydroxyapatite or the group using amine fluoride by the coordinator, who assigned random numbers generated by a computer program. However, to ensure that both the people conducting the experiment and the test subjects were blind to the product, all toothpaste tubes were identically packaged and coded by the producing/packaging company. After randomization, the four block-bearing in situdevices derived from one tooth were assigned to one test subject. Then, the first of the four assigned devices was fastened on the buccal surface of the selected lower molar by a qualified dentist in accordance with accepted principles of orthodontic practice. To fasten the device, the buccal surface of the selected tooth was gently etched for 30 seconds, washed with water, dried for 30 seconds, and isolated using cotton rolls. The lower side of the device was coated with Transbond™ XT easy curing adhesive paste (3M Unitek, Monrovia, Calif., USA) and carefully placed. The excess material emerging from the sides was used to cover the sides and the adhesive paste was cured for 20 seconds using an Ortholux XT (3M Unitek, Monrovia, Calif., USA). After fastening the device, each test subject was given his/her appropriate test toothpaste and a special soft toothbrush. Subjects were instructed to continue their routine of brushing their teeth twice a day for two minutes using only 10 milliliters of water for rinsing. In addition, special instructions were given for dispensing the toothpaste, and test subjects were asked not to brush the device directly, not to eat or drink for at least 30 minutes after brushing, and not to use other oral hygiene products (e.g., mouthwash, chewing gum etc.). As a control, a diary was provided to each test subject to record the time of each brushing phase, and the weight of the toothpaste tubes was determined. After two weeks, without using the test toothpaste that morning, the test subjects had the device removed and sent to the laboratory for analysis. The device for the second two-week treatment phase was fastened. Upon completion of the second two-week treatment phase, the second device was removed from the test subjects and they were given a rinse toothpaste and soft-bristled toothbrush, so that they could undergo a seven-day rinsing phase without the device to prepare for phase 2 of the study. After completing this rinsing phase, the phase 1 procedure was repeated to complete the second two-week treatment, so that each test subject had passed through both arms of the study.

Post-Study Procedure and Study Exit

After inter-oral exposure, a section approximately 150 μm thick was cut from each healthy and lesion-containing tooth block and processed for microradiography as previously described for baseline control sections. Although the lesion-containing control sections were microradiographed to select appropriate lesions, they were microradiographed again together with post-test sections to quantify Az and LD of the lesions, as with the baseline sections. This step allows control and test sections from the same block to be microradiographed and analyzed under the same conditions. For the lesion-containing sections, this process yielded the mineral loss (Δz₁) and lesion depth (LD₁) before the test, the mineral loss (Δz₂) and lesion depth (LD₂) after the test, and the microradiograms for the lesions before and after the test. For the healthy sections, this process yielded the mineral loss (Δz) and lesion depth (LD) after the test and the microradiograms before and after the test. Using the microradiographs, the pattern and extent of remineralization in each lesion which was produced by the treatment by each treatment arm was examined by comparing the images before and after the test. For each test subject, the mineral loss after treatment was subtracted from the mineral loss before treatment, and then standardized among the test subjects by dividing this difference by the mineral loss before treatment to obtain the remineralization in percent. The depth of lesions before and after treatment were managed in the same way to obtain the reduction in lesion depth in %. The two toothpastes used were compared using these values.

Analysis and Calculation of Sample Size

Sample size calculations were performed using nQuery Advisor software (Statistical Solutions, Cork, Ireland). Based on previous studies in which the mean percent remineralization was 30.3 with a standard deviation of 16.3 and assuming that each of the two toothpaste compositions promotes remineralization and a reduction in lesion depth significantly greater than zero, an effective sample size of 30 test subjects has an informative power of 0.95 with a one-tailed 0.05 significance level. A difference between a hypothetical mean of zero and a sample mean of remineralization equal to or greater than 10% can be determined hereby using a two-tailed t-test of two independent means. To provide a 5% failure, however, 32 test subjects were included.

Statistical Analysis

Three endpoints were determined in each case to determine mineral loss and lesion depth (1). The mean amount of remineralization and mean amount of lesion depth reduction was determined for the hydroxyapatite-containing toothpaste (Karex™) as a respective percentage of mineral loss before treatment and lesion depth before treatment. These percentages were compared to a value of 0, which is the expected value of a toothpaste with no effect. The statistical test used for this purpose was a one-tailed t-test of a group mean. (2) In the same way, the mean amount of remineralization and the mean amount of lesion depth reduction for the amine fluoride-containing toothpaste (Elmex) were determined and also compared to 0. (3) The primary endpoint was taken using the two-tailed t-test of two independent means to compare the mean of the hydroxyapatite-containing toothpaste (Karex™) to the mean of the amine fluoride-containing toothpaste (Elmex). Equivalence was established when the difference between the two toothpaste compositions was considered clinically irrelevant and Δ≤20% for each measurement method, wherein the statistical package R, version 3.5.0 was used for the analysis.

Results

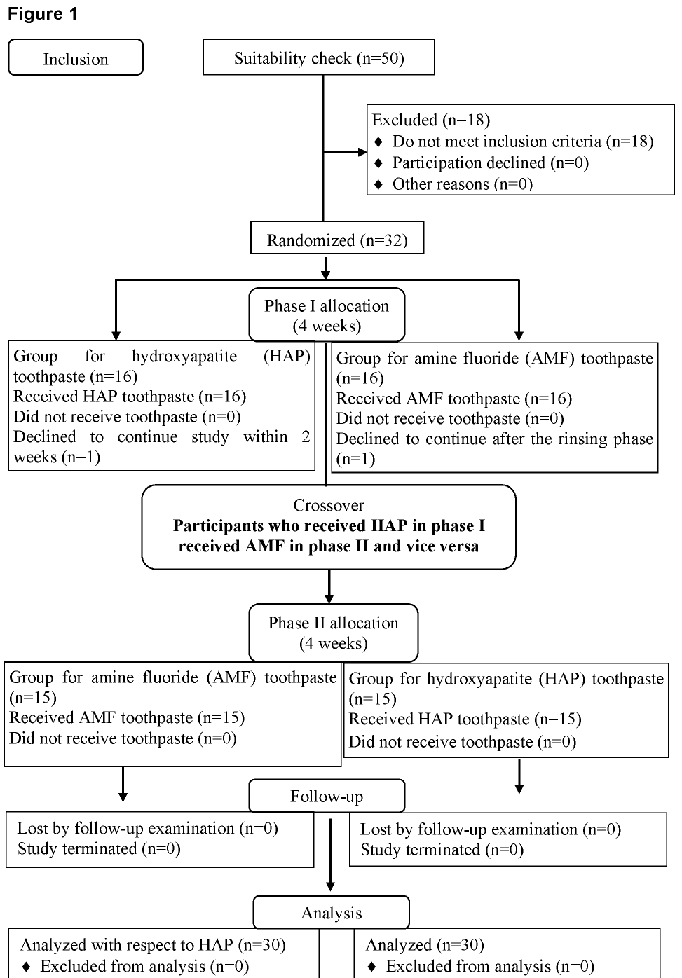

As can also be seen from FIG. 1, two test subjects dropped out of the study, one during/after the rinsing phase and one in the first two-week treatment phase while wearing the device. The study was completed by 30 subjects (19 female and 11 male) of various ethnic origins having an average age of 39.5 years.

The mean rate of rem ineralization and lesion depth reduction is shown in Table 1 below.

TABLE 1
Mean rates of remineralization and lesion depth
reduction in % for each toothpaste

| | | 500 ppm | p-value, |
|---|---|---|---|
| | 10% HAP | amine fluoride | 2 mean |
| Measurement | (Karex ™) | (Elmex) | values |
| remineralization (%) | 55.8 | 56.9 | 0.81 |
| | (SD 13.8) | (SD 14.9) | |
| p-value, a group | <0.0001 | <0.0001 | |
| lesion depth reduction | 27.1 | 28.4 | 0.68 |
| (%) | (SD 10.6) | (SD 9.8) | |
| p-value, a group | <0.0001 | <0.0001 | |

As can be seen from the table above, each of the toothpastes shows a remineralization greater than 50% and a lesion depth reduction of more than 25%. For both toothpastes, the mean remineralization and mean lesion depth reduction was statistically significantly greater than 0. When compared with one another, there was no statistically significant difference in remineralization (p=0.81) or lesion depth reduction (p=0.68). The 95% confidence interval of the difference between HAP (KarexTM) and amine fluoride (Elmex) for the mineralization was −8.8% to 6.5% and the 95% confidence interval of the difference between HAP (Karex™) and amine fluoride (Elmex) for lesion depth reduction was -6.8% to 4.1%. Consequently, this study confirms that a HAP-containing toothpaste is not inferior in effectiveness to a fluoride-containing toothpaste.

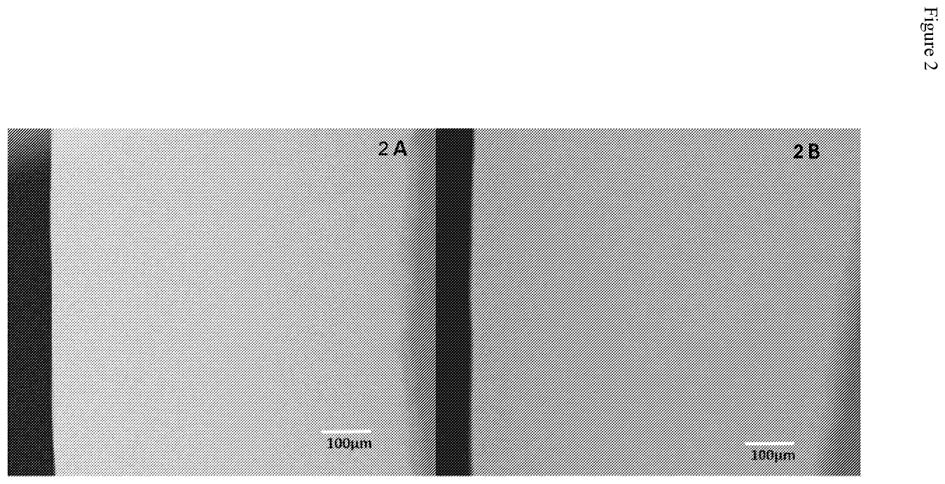

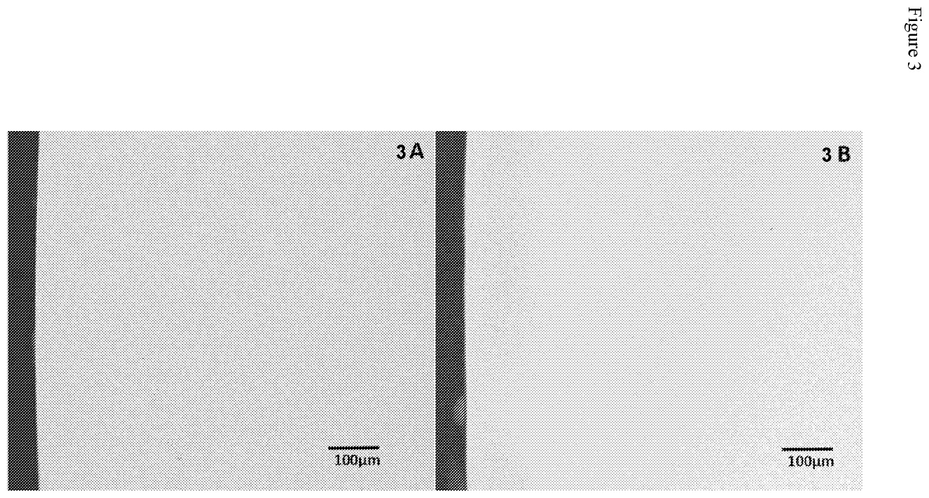

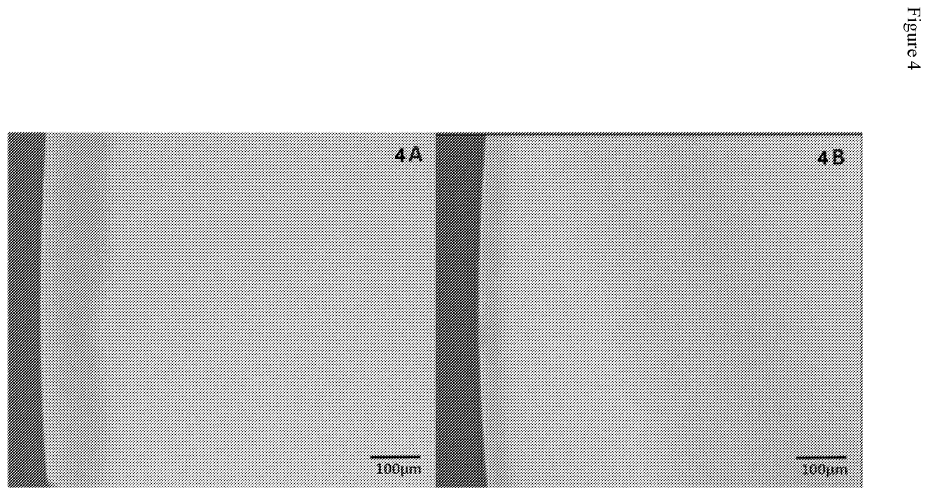

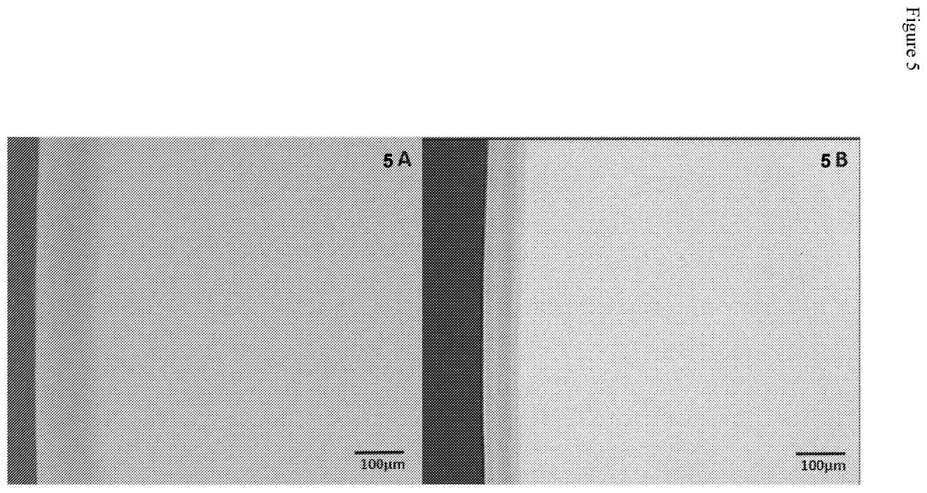

Upon analysis of the healthy tooth blocks evaluated for the ability of the two toothpastes to prevent demineralization of healthy tooth surfaces, there was no evidence of demineralization in any of the tooth blocks after intra-oral exposure to either toothpaste, as shown in FIGS. 2A & 2B as well as FIGS. 3A & 3B. In a differentiated comparison of the microradiographs of the lesion-containing specimens which were exposed during the treatment to the toothpastes having HAP (FIGS. 4A & 4B) and amine fluoride (FIGS. 5A & 5B), to the corresponding control microradiograms, the following could be established. While the HAP-containing toothpaste caused a more homogeneous remineralization throughout the thickness of the lesion lying under the surface (FIG. 4B), the remineralization caused by the amine fluoride-containing toothpaste was denser in the outer half (surface region), so that in FIG. 5B two regions having different density can be clearly seen. Overall, no incidents of adverse effects were reported by the test subjects or clinically noted.

In summary, it can be stated that a HAP-containing toothpaste is on a par with a fluoride-containing toothpaste in terms of remineralization and lesion depth reduction, but without the mentioned negative side effects that can be associated with the use of a fluoride-containing toothpaste.

In addition, unlike a fluoride-containing toothpaste, which prevents demineralization and causes remineralization in the surface region to about 30 μm, a HAP-containing toothpaste can be used to prevent demineralization and also to remineralize subsurface regions, for example in the shown case of approximately 100 μm. It was thus shown under conditions of the oral cavity (in situ) that, in particular, deeper demineralized/carious tooth enamel areas are homogeneously rem ineralized. This can improve the resistance of the teeth to dental diseases.

1-10. (canceled)

11. A method for the remineralization of teeth, said method comprising administering a composition comprising Ca₅(PO₄)₃(OH) (hydroxyapatite; HAP) to a surface of one or more teeth, wherein administration of the composition provides deep-layer remineralization to the one or more teeth.

12. The method of claim 11, wherein the composition is free of fluoride.

13. The method of claim 11, wherein the composition comprises Ca₅(PO₄)₃(OH) in an aggregated form.

14. The method of claim 11, wherein the composition comprises Ca₅(PO₄)₃(OH) in an microaggregated form.

15. The method of claim 11, wherein the composition comprises Ca₅(PO₄)₃(OH) in an amount of 0.1 to 50.0 wt.c/o in relation to the total weight of the composition.

16. The method of claim 11, wherein the composition comprises Ca₅(PO₄)₃(OH) in an amount of 0.1 to 20.0 wt.c/o in relation to the total weight of the composition.

17. The method of claim 11, wherein the one or more teeth are remineralized up to a depth of 200 μm.

18. The method of claim 11, wherein the one or more teeth are remineralized up to a depth of 100 μm.

19. The method of claim 11, wherein the composition is administered to improve a cosmetic or medical condition of the one or more teeth.

20. A method for treating a disease, said method comprising administering a composition comprising Ca₅(PO₄)₃(OH) (hydroxyapatite; HAP) to a surface of one or more teeth of a subject in need thereof,
wherein the disease comprises caries, tooth erosion, tooth wear, tooth abrasion, bruxism, molar incisor hypomineralization (MIH), amelogenesis imperfecta, dentinogenesis imperfecta, or fluorosis, and wherein administration of the composition provides deep-layer remineralization to the one or more teeth.

21. The method of claim 20, wherein the caries is a code 3 or code 4 caries according to the International Caries Detection and Assessment System (ICDAS).

22. The method of claim 20, wherein the composition comprises Ca₅(PO₄)₃(OH) in an aggregated form.

23. The method of claim 20, wherein the composition comprises Ca₅(PO₄)₃(OH) in an microaggregated form.

24. The method of claim 20, wherein administration of the composition remineralizes the one or more teeth up to a depth of 200 μm.

25. The method of claim 20, wherein administration of the composition remineralizes the one or more teeth up to a depth of 100 μm.

26. The method of claim 20, wherein the composition comprises Ca₅(PO₄)₃(OH) in an amount of 0.1 to 50.0 wt.c/o in relation to the total weight of the composition.

27. The method of claim 20, wherein the composition comprises one or more pharmaceutical or cosmetic ingredients and/or is free of fluoride.

28. A dental care composition for the remineralization of teeth, wherein said composition comprises Ca₅(PO₄)₃(OH) (hydroxyapatite; HAP), one or more pharmaceutical or cosmetic ingredients, and is free of fluoride, and wherein the dental care composition is able to provide deep-layer remineralization to the teeth.

29. The dental care composition of claim 28, the composition comprises Ca₅(PO₄)₃(OH) in an amount of 0.1 to 50.0 wt.c/o in relation to the total weight of the composition.

30. The dental care composition of claim 28, wherein the dental care composition is able to remineralize teeth up to a depth of 200 micrometers.
